(19) United States
(12) Patent Application Publication
(10) Pub. No.: US 2022/0354112 A1
(43) Pub. Date: Nov. 10, 2022

(54) DECONTAMINATION COMPOSITION AND PROCESS

(71) Applicant: William R. Martin, Eastanollee, GA (US)

(72) Inventor: William R. Martin, Eastanollee, GA (US)

(21) Appl. No.: 17/314,742

(22) Filed: May 7, 2021

(51) Int. Cl. A01N 25/16; A61L 2/22; A01N 59/20; A01N 59/16

(52) U.S. Cl. CPC A01N 25/16; A61L 2/22; A01N 59/20; A01N 59/16; A61L 2202/15; A61L 2202/25

(57) ABSTRACT

A process is described utilizing a composition of nano particles to form a foam and applying the foam to a surface to be disinfected.

### FIELD OF THE INVENTION

The present invention relates generally to the art of decontamination and more particularly to a composition useful for decontaminating surfaces having pathogens and/or particulate matter thereon.

### BACKGROUND

The removal of materials from areas such as housing, buildings and construction sites is often dangerous. If the buildings have surfaces being treated or removed containing pathogens and/or objectionable particulate matter such as mold or asbestos subject to becoming airborne, such airborne materials may be inhaled resulting in illnesses and/or ingestion of objectionable materials. The presence of mold or mold spores, asbestos and pathogens, can create extremely hazardous conditions.

When mold is present it is comprised of tiny spores of particulate matter which reproduce. These tiny spores travel through the air upon the smallest amount of movement and when the mold spores land on damp spots, these spores begin growing and digesting whatever they are growing in order to survive. Mold growth also occurs and becomes particularly troublesome when excessive moisture or water accumulates and remains undiscovered or unaddressed for long periods of time.

The same is true for pathogens such as viruses and gram-negative of bacteria. Currently there is a pandemic associated with the Corona Virus identified as Covid-19. If these pathogens are not eliminated or disinfected they spread and cause significant illness within the population.

Because of these health effects particulate matter removal and pathogen removal or disinfection is necessary. During removal it is critical that measures be taken to minimize dust and pathogens spreading and properly contained.

U.S. Pat. No. 7,445,676 which is herein incorporated by reference extensively describes techniques for utilizing foam to remove particulate matter.

U.S. Patent Publication 2017-0312732 describes in detail utilizing nano particles for sterilization and has an extensive discussion of prior art. Such publication is herein incorporated by reference.

### SUMMARY OF THE INVENTION

It is thus an object of this invention to provide a disinfecting composition for treatment of surfaces having pathogens thereon, as well as surfaces that also contain objectionable particulate matter such as mold or asbestos.

This as well as other objects are achieved utilizing a foam composition of nano particles of copper, silver or gold with a foam precursor and expansion gas and a surfactant for the formation of the foam to be placed on contaminated surfaces.

### DETAILED DESCRIPTION

The present invention is specific to the use of foam which is applied to surfaces for decontamination and/or removal of particulate matter. The particulate matter or material containing particulate matter and/or pathogens have foam applied to such surfaces to prevent objectionable matter from becoming airborne. Foam easily conforms and migrates to newly exposed surfaces to prevent any objectionable matter from becoming airborne. Foam also has the advantage of covering a large area with a minimum amount of material. Neither a baffle attachment nor a vacuum is necessary to prevent spreading of the objectionable material on the contaminated surface. Because the exposure levels are nearly zero, persons removing objectionable material are not required to wear respirators or protective clothing.

Pathogens include viruses and gram-negative bacteria which are noted for causing significant illness. Particulate matter includes small discreet masses of solid or liquid matter which may become airborne. Examples of other types of particulate matter exist and are considered part of the scope of the present invention. In many instances, pathogens and particulate matter pose health risks because they are subject to being inhaled and lodged in a person's nose, eyes, throat or lungs when they become airborne.

For purposes of the present invention, foam is defined as a non-reactive dispersion of a gas vapor in a liquid. When being utilized for removal of objectionable matter the drain time required for the foam to decompose into original liquid and gas phases should be sufficiently long to allow for the objectionable matter to be removed and transported to a contained environment without substantial loss of foam. If only pathogens are involved the drain time is not as significant with the nano particles.

A foam production system typically includes a foam precursor, an expansion gas, and a surfactant.

The foam precursor is preferably a liquid with a surface tension sufficiently low to form a foam. The foam tension is preferably below about 30 dynes per centimeter and can be lower by incorporation of suitable surfactants. Water based systems are preferable but organic based foams are most preferred. The foam preferably has a near neutral PH of around 5 to 9 and more preferably around 6 to 8. The foam is preferably thixotropic with a viscosity that is shear dependent. In the absence of sheer force the foam will not flow and can be stacked or piled to a desired depth. It is most preferred that the foam precursor be substantially non-reactive with the objectionable matter being removed. A particularly preferred foam is FiberStop foam, product code 12-530, available from Martin Industries of Eastanolle, Ga.

The expansion gas can be air, however, other expansion gasses are known to be useful such as low molecular weight hydrocarbons, nitric oxide, or carbon dioxide. Air is preferred due to the low cost, low toxicity. Air is preferred due to the low cost, low toxicity and the fact that a separate tank is not necessary. It is most important that the expansion gas be non-soluble in the foam precursor. The foam precursor may have adjuvants as known in the art including surfactants such as sodium laurl sulfate; or adhesive additives such as styrene-butadadiene polymer thickness or viscosity modifiers such as typical paint thickners, gelatin or modified starches; and others. Foam generating equipment is well documented and not particularly limited herein. The foam generating equipment has two main components. One component combines the liquid precursor with the expansion gas. The mixing is preferably very through to insure the drain time is sufficiently long and that the expansion is optimal. The mixing can be done by passing through a hose, or pipe, with sufficient flow turbulence to mix the components. Alternatively the mixture can be passed through a mixing device such as a packed bed mixture.

As used herein, gram-negative bacteria are known class of bacteria that are characterized by thin cell walls compared to gram-positive bacteria. Any gram-negative bacteria are pathogenic, and can be harmful to animals including humans.

As used herein, gram-positive bacteria are known bacteria that are usually characterized by thick cell walls. Most gram-positive bacteria are not pathogenic and, therefore, are not harmful to humans. Copper, silver and gold, nano-particles can be synthesized by known methods. See for example, Synthesis, characterization and anti microbial properties of copper nano particles, International Journal of nano medicines, 2013: 8 pp 4467-4479. While this invention is directed to foam having nano particles of copper, silver and gold, as used herein, emphasis will be directed to copper since it is much more available than the precious metals.

While copper is naturally found as a trace metal in the environment, too much copper can be toxic, however copper nano particles can also be toxic to bacteria and viruses. While copper particles are toxic at concentrations of about 195 parts per million, copper ions are toxic at a concentration of about 15 parts per billion.

Size, shape and surface of nano particles have a significant effect on the properties of the nano particles. By altering the temperature of the initial reaction, the quantity of reactants and capping agents, specific sizes of nano particles can be synthesized. The smaller the nano particles, the larger the total surface area. Thus allowing for a greater oxidation and a greater toxicity to bacteria and other pathogens. For copper nano particles, it is believed that the optimal size for use is between 5 nano-meters and 100 nano-meters. If the composition is being used only for pathogen disinfection, the drain time can be significantly shorter since the nano particles disinfect quickly upon contact. Alcohol can be added to expedite drain time.

As defined herein, polymer-matrix nano composites consist of a polymer having nano-particles disbursed on or in the polymer. Polymer nano-deposits can serve to amplify the properties of the nano-particles by allowing for the use of smaller nano-particles, and therefore, increasing the surface area to volume ratio. Polymer-nano composites can also serve to disinfectant are less susceptible to oxidation. Some known uses of polymer-matrix nano particles include tissue engineering drug delivery and immobilization of protein. Such polymer matrix is also believed to strengthen the foam.

Cellulose, is a preferred polymer. Cellulose has been used to make paper and cardboard. It is insoluble in water and is made of betaglucose monomers. As a polymeric fiber it can act as efficiently as the polymer matrix in a nano composite. The cellulose fibers can link together forming a “cloud” of nano particles, thus entrapping them for use in disinfection while preventing leaching. For copper nano-particles cellulose based composite it is believed that the optional size for nano cloud composites used in eliminating harmful bacteria while maximizing retention is between about 2,000 nano meters and 5,000 nano meters.

A preferred concentration of nano particles in the foam precursor is preferably about 0.3 to 2 milligrams per milliliter. A preferred composition within that range is about 1.2 milligrams per milliliter.

A typical foam composition is prepared by mixing nano-particles, a foam precursor and a surfactant into a foaming device. An expansion gas is applied to the foam producing device to result in the dispensing of foam onto surfaces having objectionable matter thereon. A typical composition would contain 10 grams of nano particles, 160 grams of precursor and 40 grams of surfactant.

In cases of pathogen disinfect, foam is applied to the affected surface by spraying. After a short residence time, on the order of five minutes, the foam and disinfected pathogenic is simply rinsed off with water.

Having generally described the invention in non-limiting details, the spirit and scope of the invention is defined by the following appended claims.

1. A disinfectant foam composition capable of disinfecting and coating surfaces having a pathogen thereon, comprising:
nano particles of copper, silver or gold;
a foam precursor;
an expansion gas; and
a surfactant.

2. The composition according to claim 1 wherein said surface further has particulate matter thereon.

3. The composition according to claim 1 wherein said pathogen is a virus.

4. The composition according to claim 1 wherein the pathogen is a germ negative bacteria.

5. The composition according to claim 2 wherein said particulate matter is asbestos.

6. The composition according to claim 2 wherein said particulate matter is mold.

7. The composition according to claim 1 wherein said nano particles are of a size between 5 and 100 nano meters.

8. A process for disinfecting a surface having pathogens thereon, comprising mixing nano particles of copper, silver or gold with a foam precursor and a surfactant to form a liquid mixture;
applying an expansion gas to the liquid mixture to form a foam;
applying said foam to said surface to disinfect the pathogens thereon.

9. The composition according to claim 1, wherein concentration of nano particles in the disinfectant foam composition is about 0.3 to 2 milligrams per milliliter.

10. A disinfectant foam composition capable of disinfecting and coating surfaces having a pathogen thereon, consisting essentially of:
nano particles of copper, silver or gold;
a foam precursor;
an expansion gas; and
a surfactant.

11. (canceled)
